(19) United States
(12) Patent Application Publication
(10) Pub. No.: US 2010/0274319 A1
(43) Pub. Date: Oct. 28, 2010

(54) CURRENT LEAKAGE DETECTION FOR A MEDICAL IMPLANT

(75) Inventor: Werner Meskens, Opwijk (BE)

Correspondence Address: Connolly Bove Lodge & Hutz LLP, Suite 1100, 1875 Eye Street, NW, Washington, DC, 20006, US

(73) Assignee: COCHLEAR LIMITED, Lane Cove (AU)

(21) Appl. No.: 12/769,389

(22) Filed: Apr. 28, 2010

(30) Foreign Application Priority Data
Apr. 28, 2009 (AU) 2009901835

(51) Int. Cl. A61F 11/04; A61N 1/08; A61N 1/36

(52) U.S. Cl. 607/57; 607/63

(57) ABSTRACT

Current leakage detection techniques in an implantable medical device are disclosed. In these techniques, a core surrounds conductors carrying current to and from an implanted medical device. A secondary winding on the core picks up imbalances between the current flows on the conductors traveling through the core. An imbalance is detected if the current on the secondary winding results in a specified threshold being exceeded. Corrective action may then be taken if a current imbalance is detected.

### CROSS-REFERENCE TO RELATED APPLICATIONS

This application claims priority to commonly owned and co-pending Australian Provisional Patent Application No. 2009901835, entitled “LEAKAGE CURRENT DETECTION FOR A MEDICAL IMPLANT,” filed Apr. 28, 2009, the contents of which are hereby incorporated by reference.

This application is related to PCT Application No. PCT/AU2009/000853 entitled “POWER CONTROL FOR A MEDICAL IMPLANT,” PCT Application No.: PCT/AU96/00403, entitled “APPARATUS AND METHOD OF CONTROLLING SPEECH PROCESSORS AND FOR PROVIDING PRIVATE DATA INPUT VIA THE SAME,” and PCT Application No. PCT/AU2009/000843, entitled “SOUND PROCESSOR FOR A MEDICAL IMPLANT.” The content of these applications are hereby incorporated by reference herein.

### BACKGROUND

1. Field of the Invention

The present invention relates generally to an implantable medical device, and more particularly to a current leakage detection system for an implantable medical device.

2. Related Art

A variety of implantable medical devices have been proposed to deliver controlled electrical stimulation to a region of a subject's body to perform a desired function. One such device is a heart pacer, also referred to as a pacemaker, which uses electrical impulses, delivered by electrodes contacting the heart muscles, to regulate the beating of the heart. Another such device which has been successful in providing hearing sensation to individuals with sensorineural hearing loss is the cochlear implant. For individuals with sensorineural hearing loss, there is typically damage to or an absence of hair cells within the cochlea which convert acoustic signals into nerve impulses which are perceived as sound by the brain. Such individuals are unable to derive suitable benefit from conventional hearing aid systems, and hence look to rely upon cochlear implants to provide them with the ability to perceive sound.

Cochlear implants use electrical stimulation of auditory nerve cells to bypass absent or defective hair cells that normally transduce acoustic vibrations into neural activity. Such devices generally use an array of electrode contacts implanted into the scala tympani of the cochlea so that the stimulation may differentially activate auditory neurons that normally encode differential frequencies of sound.

Auditory brain stimulators are used to treat a smaller number of recipients with bilateral degeneration of the auditory nerve. For such recipients, the auditory brain stimulator provides stimulation of the cochlear nucleus in the brainstem. Auditory brain stimulators similarly use a plurality of electrode contacts to provide stimulation to the recipient.

Engineers and technicians have, with improvements in technology and knowledge, been making the devices smaller and therefore more readily implantable. Improvements to functions and the increased complexity of devices and functions are an important part of the progressive development of implantable devices. However, as implantable devices become increasingly complex, the potential for electrical failures increases.

Such failures can result in current leakage, with the excess current passing through tissue of the implantee in ways which are not related to therapy. Such currents flows could result in electrolysis, or otherwise cause injury to the user. Currents can also cause irreversible redox reactions at the electrodes of the implanted device that may result in toxic products near the electrode and/or pH changes in the tissue.

By way of example, current cochlear implants are capable of detecting fault conditions in only a very limited way, usually by regularly checking for particular faults. The faults being checked for are programmed into the implant based on the failure modes determined by the design team. For example, electrodes may short to ground. As devices become more complex, the number of failure modes that can lead to DC current leakage increases dramatically. As such, the present methods of checking for faults will take an increasing amount of time and power, and be increasingly complex to design and operate. Further, it becomes increasingly difficult to determine all possible failure modes, and to try to detect each specific failure mode.

### SUMMARY

In one aspect of the present invention an implantable medical device is provided. The implantable medical device comprising: at least one electronic circuit; a current imbalance detector; and a hermetically sealed housing that houses said at least one electronic circuit and said current imbalance detector. The current imbalance detector comprises a core surrounding at least a portion of one or more electrical conductors connected to the at least one electronic circuit; a winding on the core; and a detection circuit connected to the winding and configured to provide a signal indicative of whether there is an imbalance in current conducted by the one more electrical conductors.

In another aspect, there is provided a method for use in an implantable medical device having at least one electronic circuit. The method comprises obtaining a signal representative of a sensed signal from a winding on a core, wherein the core surrounds at least a portion of one or more electronic conductors connected to the at least one electronic circuit of the implantable medical device; determining if the sensed signal indicates an imbalance in current conducted by the one or more electrical conductors; and performing a corrective action if a current imbalance is detected exceeding a threshold.

In yet another embodiment, there is provided a system for use in an implantable medical device having at least one electronic circuit. The system comprises: means for obtaining a signal representative of a sensed signal from a winding on a core, wherein the core surrounds at least a portion of one or more electrical conductors connected to the at least one electronic circuit of the implantable medical device; means for determining if the sensed signal indicates an imbalance in current conducted by the one or more electrical conductors; and means for performing a corrective action if a current imbalance is detected exceeding a threshold.

### BRIEF DESCRIPTION OF THE DRAWINGS

Embodiments of the present invention are described below with reference to the attached drawings, in which:

FIG. 1 is a perspective view of a cochlear implant in which embodiments of the present invention may be implemented;

FIG. 2 is a functional block diagram of the cochlear implant of FIG. 1, in accordance with an embodiment of the invention;

FIG. 3 is an exemplary current implant leakage model and is provided to illustrate possible current leakage paths that may exist in an implanted device;

FIG. 4 illustrates a range of external sources that can affect an implant;

FIG. 5 is a schematic overview of an internal component of a cochlear implant comprising a current leakage detection system, in accordance with an embodiment of the present invention;

FIG. 6 provides a more detailed illustration of an exemplary leakage detection circuit, in accordance with an embodiment of the invention;

FIG. 7 illustrates a leakage detection system comprising a ferrite core having a cylindrical shape, in accordance with an embodiment of the invention;

FIG. 8 provides a high level flow chart illustrating operations that may be performed in detecting a current leakage, in accordance with an embodiment;

FIG. 9A illustrates leakage detection circuit along with arrows pointing to various points, A-F, along leakage detection circuit, in accordance with an embodiment of the invention;

FIG. 9B illustrates the signals at the points illustrated in FIG. 9B during a situation in which a current imbalance exists due to current leakage, in accordance with an embodiment of the invention;

FIG. 10 is a schematic overview of an internal component of a cochlear implant system comprising a current leakage detection system on wires passing between a main implant unit and a secondary coil, in accordance with an embodiment of the present invention

FIG. 11 provides a simplified illustration of a system capable of providing low pass filtering, in accordance with an embodiment of the invention;

FIG. 12 illustrates an exemplary embodiment of a stimulator unit comprising a leakage detection system in combination with a balun, in accordance with an embodiment of the invention;

FIG. 13 illustrates is a schematic overview of an internal component of a cochlear implant system comprising a current leakage detection system on wires passing between the main implant unit and an auxiliary implant unit, in accordance with an embodiment of the present invention; and

FIG. 14 illustrates is a schematic overview of an internal component of a cochlear implant system in which the secondary coil is located within the housing of the main implant unit, in accordance with an embodiment of the invention.

### DETAILED DESCRIPTION

Embodiments of the present invention are generally directed to current leakage detection techniques in implantable medical devices. As will be discussed in more detail below, in an embodiment, a core (e.g., a ferrite core) surrounds the conductors carrying current to and from an implanted medical device. A secondary winding on the core picks up imbalances between the current flows traveling through the core. An imbalance may be detected if the current picked up by the secondary winding exceeds a specified threshold. Corrective action may then be taken if a current imbalance is detected.

Embodiments of the present invention are described herein primarily in connection with one type of implantable medical device, a hearing prosthesis, namely a cochlear prosthesis (commonly referred to as cochlear prosthetic devices, cochlear implants, cochlear devices, and the like; simply “cochlea implants” herein.) Cochlear implants deliver electrical stimulation to the cochlea of a recipient. It should, however, be understood that the current leakage techniques described herein are also applicable to other types of active implantable medical devices (AIMDs), such as, auditory brain stimulators, also sometimes referred to as an auditory brainstem implant (ABI), other implanted hearing aids or hearing prostheses, neural stimulators, retinal prostheses, cardiac related devices such as pacers (also referred to as pacemakers) or defibrillators, implanted drug pumps, electro-mechanical stimulation devices (e.g., direct acoustic cochlear stimulators (DACS)) or other implanted electrical devices.

As used herein, cochlear implants also include hearing prostheses that deliver electrical stimulation in combination with other types of stimulation, such as acoustic or mechanical stimulation (sometimes referred to as mixed-mode devices). It would be appreciated that embodiments of the present invention may be implemented in any cochlear implant or other hearing prosthesis now known or later developed, including auditory brain stimulators, or implantable hearing prostheses that mechanically stimulate components of the recipient's middle or inner ear. For example, embodiments of the present invention may be implemented, for example, in a hearing prosthesis that provides mechanical stimulation to the middle ear and/or inner ear of a recipient.

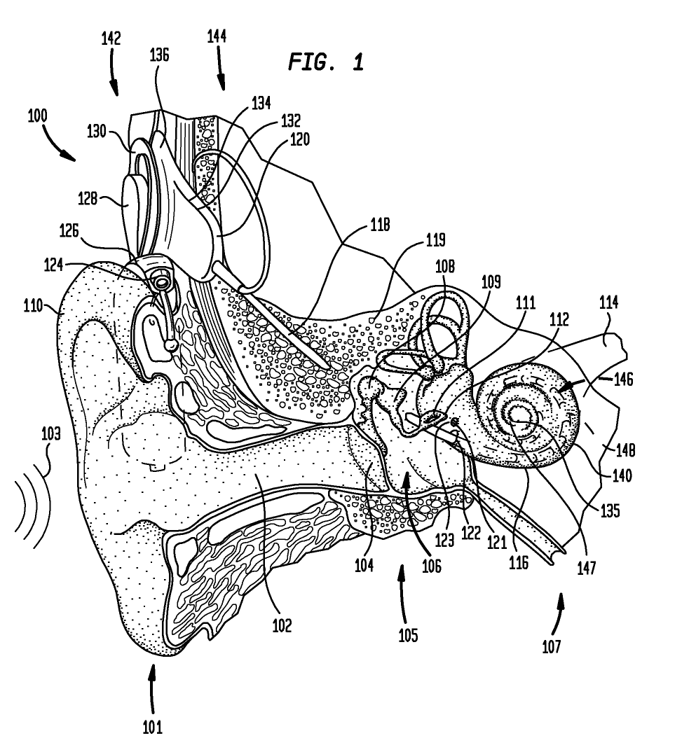

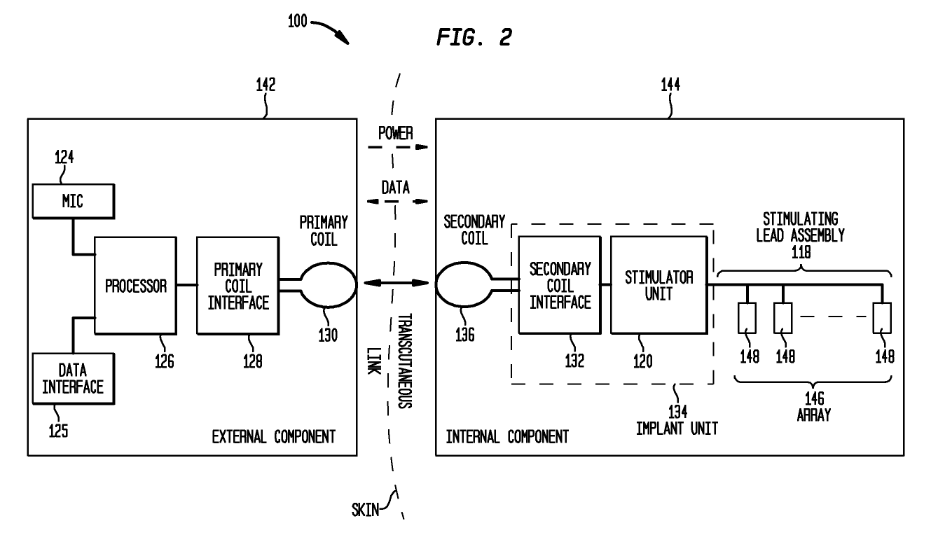

FIG. 1 is perspective view of a cochlear implant, referred to as cochlear implant system 100 implanted in a recipient. FIG. 2 is a functional block diagram of cochlear implant 100. The recipient has an outer ear 101, a middle ear 105 and an inner ear 107. Components of outer ear 101, middle ear 105 and inner ear 107 are described below, followed by a description of cochlear implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear cannel 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109 and the stapes 111. Bones 108, 109 and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

Cochlear implant system 100 comprises an external component 142 which is directly or indirectly attached to the body of the recipient, and an internal component 144 which is temporarily or permanently implanted in the recipient. External component 142 is often referred as a sound processor device that typically comprises one or more sound input elements, such as microphone 124 for detecting sound, a processor 126, a power source (not shown), and an external coil driver unit 128 (referred to herein as primary coil interface 128). External coil interface unit 128 is connected to an external coil 130 (also referred to herein as primary coil 130) and, preferably containing a magnet (not shown) secured directly or indirectly concentric to internal coil 136 (also referred to herein as secondary coil 136). External and internal coils are closely coupled enabling power and data transfers by inductive link. Processor 126 processes the output of microphone 124 that is positioned, in the depicted embodiment, behind the ear of the recipient. Processor 126 generates encoded signals, sometimes referred to herein as encoded data signals, which are provided to the external coil interface unit 128 via a cable (not shown).

The internal implant component 144 comprises an internal coil 136 (also referred to herein as secondary coil 136), an implant unit 134, and a stimulating lead assembly 118. As illustrated, implant unit 144 comprises a stimulator unit 120 and a secondary coil interface 132 (also referred to as secondary coil interface 132). Secondary coil interface 132 is connected to the secondary coil 136. Secondary coil 136 may include a magnet (also not shown) fixed in the middle of secondary coil 136. The secondary coil interface 132 and stimulator unit 120 are hermetically sealed within a biocompatible housing, sometimes collectively referred to as a stimulator/receiver unit. The internal coil receives power and stimulation data from primary coil 130. Stimulating lead assembly 118 has a proximal end connected to stimulator unit 120, and a distal end implanted in cochlea 140. Stimulating lead assembly 118 extends from stimulator unit 120 to cochlea 140 through mastoid bone 119. In some embodiments stimulating lead assembly 118 may be implanted at least in basal region 116, and sometimes further. For example, stimulating lead assembly 118 may extend towards apical end of cochlea 140, referred to as cochlea apex 147. In certain circumstances, stimulating lead assembly 118 may be inserted into cochlea 140 via a cochleostomy 122. In other circumstances, a cochleostomy may be formed through round window 121, oval window 112, the promontory 123 or through an apical turn 135 of cochlea 140.

Stimulating lead assembly 118 comprises a longitudinally aligned and distally extending array 146 of electrode contacts 148, sometimes referred to as array of electrode contacts 146 herein. Although array of electrode contacts 146 may be disposed on Stimulating lead assembly 118, in most practical applications, array of electrode contacts 146 is integrated into Stimulating lead assembly 118. As such, array of electrode contacts 146 is referred to herein as being disposed in Stimulating lead assembly 118. Stimulator unit 120 generates stimulation signals which are applied by electrode contacts 148 to cochlea 140, thereby stimulating auditory nerve 114. Because, in cochlear implant 100, Stimulating lead assembly 118 provides stimulation, Stimulating lead assembly 118 is sometimes referred to as a stimulating lead assembly.

In cochlear implant system 100, primary coil 130 transfers electrical signals (that is, power and stimulation data) to the internal or secondary coil 136 via an inductive coupled radio frequency (RF) link. Secondary coil 136 is typically made of multiple turns of electrically insulated single-strand or multi-strand platinum or gold wire. The electrical insulation of secondary coil 136 is provided by a biocompatioble wire insulator and a flexible silicone molding (not shown). In use, secondary coil 136 may be positioned in a recess of the temporal bone adjacent auricle 110 of the recipient.

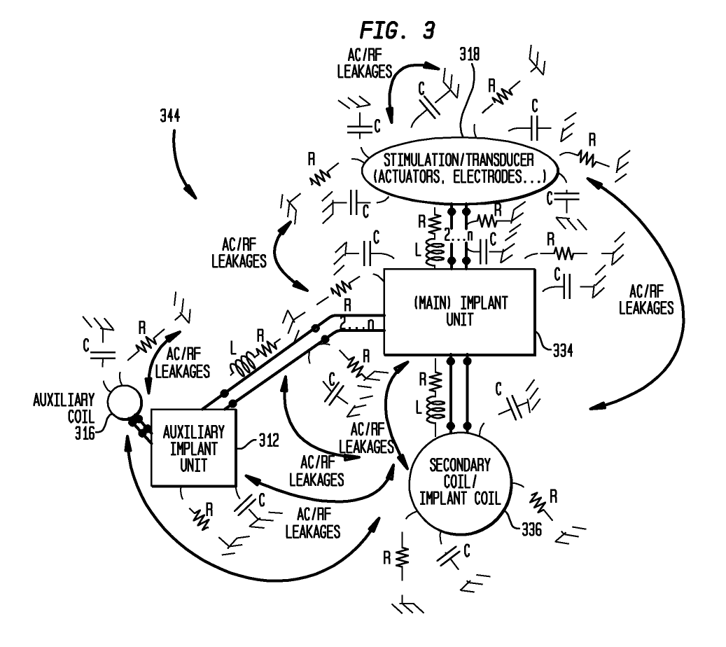

FIG. 3 is an exemplary current implant leakage model and is provided to illustrate possible current leakage paths that may exist in an implanted device. As illustrated, an implanted device 344 may comprise a main implant unit 334, a secondary coil 336, stimulation/transducer devices 318, an auxiliary implant 312 and an auxiliary coil 316. Implanted device 344 may be, for example, an internal component 144 of a cochlear implant system 100 such as discussed above with reference to FIGS. 1 and 2. For example, the main implant unit 334 may contain the coil interface 132 and stimulator unit 120, stimulation/transducer devices 318 may be a stimulation lead assembly 118, and coil 336 may be the secondary coil 136. Auxiliary implant 312 may comprise, for example, a hermetically sealed battery for providing power to implant 334. Auxiliary coil 316 may receive power via an external power source for recharging battery. In such an embodiment, the implant unit 334 may receive data via secondary coil 336 and power via auxiliary implant 312. As shown in FIG. 3, current leakage may occur in numerous pathways that have capacitance and resistance qualities. For example, as shown current leakage may occur between the auxiliary implant 312 and secondary coil 336, between the stimulation/transducer device 318 and the secondary coil 336 or auxiliary coil 316, etc.

In other embodiments, implanted device 344 may be another implantable medical device, such as an ABI, a pacemaker, FES systems, SCS systems, pacemakers or other heart stimulation devices, implantable drug-dispensing devices and bone growth stimulators. Further, the auxiliary implant 312 may be other types of devices other than a battery, such as a hermetically sealed housing comprising electronics for performing mechanical or electrical stimulation or electronics for sensing the results of applied stimulation. Or, in other embodiments, auxiliary implant unit 312 may comprise a battery and or other electronics, such as a microphone or a wireless transceiver.

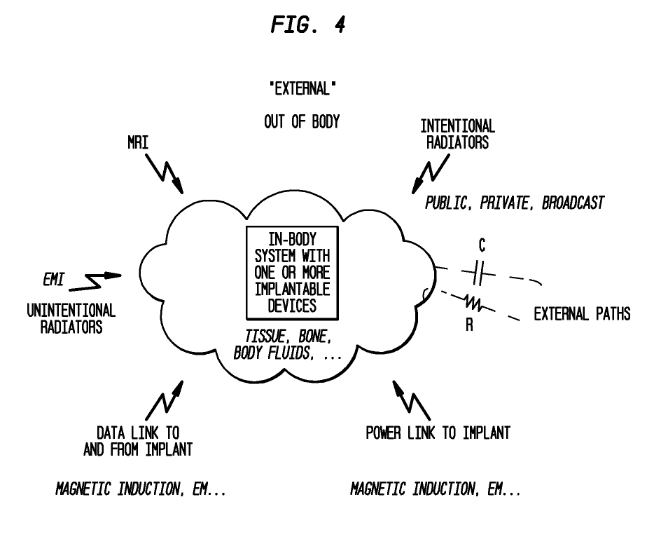

In addition to current leakage resulting from parasitic current transfer between components of the implant or to a ground, current leakage may also result from external factors, such as radiation, electrical, and/or magnetic fields invoking current through the recipient's tissue as a result of an imperfection(s) in the implant system. FIG. 4 illustrates a range of external sources that can affect an implant. As illustrated, these external fields may comprise intentional radiators (e.g., public and private broadcast communications), electromagnetic interferences (EMI), external device(s) providing power and data to the implant via, for example, by magnetic induction using alternating magnetic fields. Another example of an external field that may invoke current in the implant is a field generated by a Magnetic Resonance Imaging (MRI) scanner. MRI scanners can generate strong electromagnetic pulsed RF and magnetic fields. These external magnetic, radiation, and/or electrical fields can induce large signals on conductive wires or leads in the implant that may force the system's electronics to operate in a non-linear manner or even at saturation.

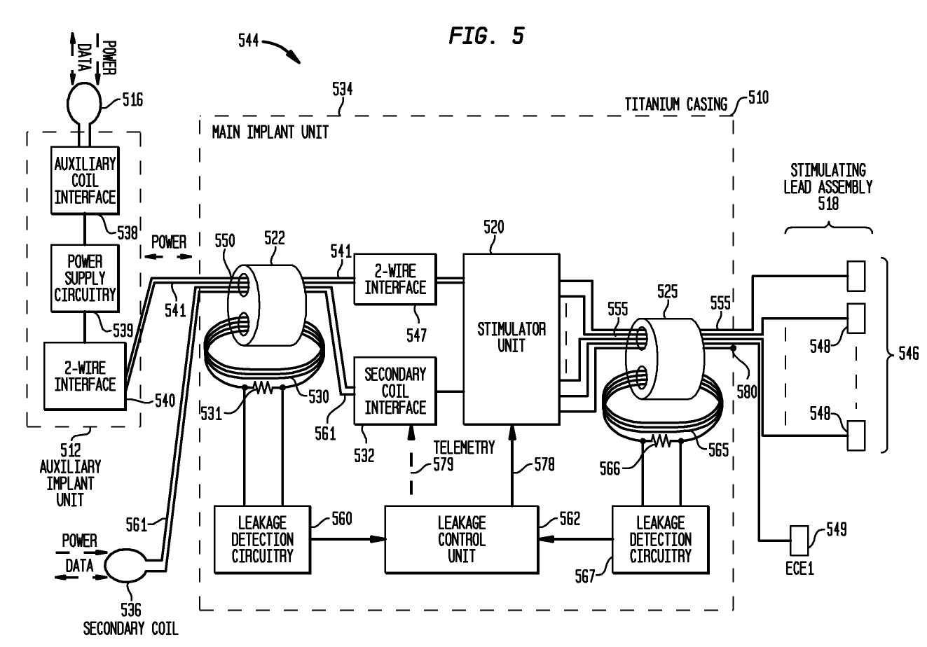

FIG. 5 is a schematic overview of an internal component of a cochlear implant comprising a current leakage detection system, in accordance with an embodiment of the present invention. The illustrated leakage detection system may help detect current leakage problems such as discussed above with reference to FIGS. 3-4. FIG. 5 will be discussed with reference to a cochlear implant, such as illustrated in FIG. 1. However, it should be understood that the leakage detection system may be implemented in other implanted medical devices as noted above.

As illustrated, internal component 544 comprises a secondary coil 536, an implant unit 534 and stimulating lead assembly 518. As shown, implant unit 534 comprises a stimulator unit 520 and a secondary coil interface 532, such as stimulator unit 120 and secondary coil interface 132 discussed above with reference to FIG. 1. As illustrated, internal coil interface 532 is connected to the internal secondary coil 536. In FIG. 5, implant unit 534 will be hereinafter referred to as main implant unit 534. Also, as illustrated, main implant unit 534 is connected to a stimulating lead assembly 518 comprising a plurality of electrode contacts 548 of an array of electrode contacts 546, such as stimulating lead assembly 118 (FIG. 1). Additionally, in this exemplary embodiment, main implant unit is also connected to a first extra-cochlea electrode 549 and a second extra-cochlea electrode 580. Each of these extra-cochlea electrodes may be manufactured from a biocompatible conductive material (e.g., platinum), and be an extra-cochlea electrode such as used in cochlear implants employing monopoloar stimulation. In the illustrated embodiment, the second extra-cochlea electrode 580 is mounted to the housing 510 of main implant unit 534. In an embodiment, extra-cochlea electrodes are not included, such as, for example, in embodiments employing bipolar, tri-polar, and/or phased array stimulation.

Additionally, in this embodiment, internal component 544 further comprises an auxiliary implant unit 512 connected to a coil 516. Auxiliary implant unit 512 may provide power to the main implant unit 534. Auxiliary implant unit 512 may comprise an auxiliary coil interface 538, a power supply circuitry 539, and a 2-wire interface 540. Power supply circuitry 539 may comprise rechargeable battery (not shown). Power may be transmitted by an external coil that is received by coil 516, which provides the received power to power supply circuit 539 for recharging the battery.

In the illustrated embodiment, two-wire interfaces 540 and 547 are used for transferring power and data between auxiliary implant unit 512 and main implant unit 534. This power and/or data transfer may be bi-directional or uni-directional. For example, in an embodiment, power may be transferred via secondary coil 536 and this power provided to auxiliary implant unit 512 from main implant unit 534 via two-wire interfaces 547 and 540. Although in the illustrated embodiment two-wire interfaces 540 and 547 connect auxiliary implant unit 512 and main implant unit 534, in other embodiments additional wires may be used. For example, auxiliary implant unit 512 may output power having different voltage levels on different wires, or other wires may be used to carry data, such as data from a microphone included in auxiliary implant unit 512.

As noted above, each of auxiliary implant unit 512 and main implant unit 534 may be encapsulated in a hermetically sealed biocompatible housing 510, such as, for example a titanium housing. Or, for example, in an embodiment, housing 1410 may be manufactured from an organic polymer thermoplastic such as polyether ether ketone (PEEK). Or, for example, housing 1410 may be a ceramic housing.

Stimulator unit 520 may comprise one or more integrated circuits for receiving the stimulation data transmitted by the external component (e.g., external component 142 of FIG. 1) and providing stimulation in accordance with the received stimulation data via the electrodes 548, such as was discussed above with reference to FIG. 1.

Further as shown, the main implant unit 534 comprises a front-end leakage detection system comprising a core 522 through which incoming/outgoing wires 550 pass, a winding 530, a sense resistor, Rₛₑₙₛₑ, 531 and a leakage detection circuit 560. As shown, wires 550 comprises wires 541 and wires 561. Wires 541 connect a 2-wire interface 547 in main implant unit 534 to the 2-wire interface 540 of auxiliary implant unit 512. Wires 561 connect secondary coil interface 532 to secondary coil 536.

Also, as shown, main implant unit 534 also comprises a back-end leakage detection system comprising a core 525, through which incoming/outgoing wires 555 pass, a winding 565, a sense resistor, Rₛₑₙₛₑ, 566 and a leakage detection circuit 567. Wires 555 comprise wires connecting stimulator unit 520 to electrode contacts 148. In cochlear implants employing an extra-cochlear electrode 549, wires 555 may also comprise any wires connecting stimulator unit 120 to extra-cochlea electrode 549. Additionally, as shown wires 555 may comprise a wire connecting stimulator unit 520 to the housing 580 of main implant unit 534. As noted above, main implant unit 534 may be encapsulated in a hermetically sealed housing, such as for example, a hermetically sealed titanium casing.

The embodiment of FIG. 5 will be discussed with reference to cores 522 and 525 each being a ferrite core. It should be understood, however, that as will be discussed in further detail below, in other embodiments cores 522 and 525 may be a different type of core and/or have a different shape than illustrated. As used herein, the term “core” refers to a component serving as a part of a path for magnetic flux, such as, for example, a transformer's core. Exemplary cores include, for example, ferromagnetic and ferrimagnetic cores, such as, ferrite cores, laminated steel cores, silicon steel cores, powdered iron cores, etc.

For ease of explanation, the operation of the front-end leakage detection system will be initially described. After which, the operation of the back-end leakage detection system, which operates similarly, will be discussed. As noted, wires 550 connect auxiliary implant unit 512 and secondary coil 536 to main implant unit 534. Wires 550 carry power and/or data to stimulator unit 520 of main implant unit 534. Further, as illustrated, winding 530 also passes through ferrite core 522 and is connected to sense resistor, Rₛₑₙₛₑ, 531. As is known to those of skill in the art, the turns ratio for a winding is equal to the ratio of the number of turns in the secondary winding to the number of turns of the primary winding. In this example, winding 530 comprises N turns around ferrite core 522 and wires 550 form one turn around ferrite core 522. Thus, the turn ratio for winding 530 is equal to N.

During normal operations in the present embodiment, the current passing into main implant unit 534 via wires 550 should equal the current exiting main implant unit 534. If the sum of incoming and outgoing currents is different from zero, then current leakage may exist. Thus, the principle of the presently described current leakage detection system is that if everything is working correctly, the same current should be flowing into the main implant unit 534 and through ferrite core 522 as is passing out of main implant unit 534 via ferrite core 522. In other words, if everything is working properly, the sum of all currents through the wires 550 passing through ferrite core 522 should be equal to zero. Any difference is indicative of current flowing into or out of the tissue from unknown paths, which is indicative of a fault of some kind.

This system takes advantage of Kirchhoff's current law in which the sum of currents flowing towards a point in a circuit is equal to the sum of currents flowing away from that point. Due to Kirchoff's current law, the total sum of currents entering the tissue in an implanted device is zero. If the sum of current entering along known paths is not zero, then the left-over current must be entering the tissue along an unknown or fault path.

In the illustrated embodiment, the voltage, Vₛₑₙₛₑ, over the sense electrode, Rₛₑₙₛₑ, 531, is in direct relationship with the turn ratio N and the common mode current, I_{common}, which is the sum of the current on wires 550. Particularly, in the illustrated example, Vₛₑₙₛₑ=Rₛₑₙₛₑ*I_{common}*N. Thus, if common mode current, I_{common}, is large due to current leakage, the resulting sensed signal (e.g., sensed voltage, Vₛₑₙₛₑ), will likewise be relatively large. Or, if there is no or minimal current leakage, then the sensed voltage, Vₛₑₙₛₑ, will be respectively zero or comparatively low. As such, in the illustrated embodiment, the sensed voltage, Vₛₑₙₛₑ, provides an indication of the magnitude of any current leakage that may exist in internal component 544. As noted above with reference to FIGS. 3 and 4, this detected current leakage may result from leakage of current by the implant's components or current leakage resulting from externally generated fields.

The sensitivity of leakage detection circuitry 560 may be controllable. For example, increasing the number of turns, N, of secondary winding 530 increases the voltage over the resistor, Rₛₑₙₛₑ. The resistance of the resistor, Rₛₑₙₛₑ, may be chosen to be very high, and in certain embodiments may be removed so as to effectively provide an infinite resistor value. However, noise voltage may be related to bandwidth and resistance by the following equation: Vₙₒᵢₛₑ˜=sqrt(4kTBR), where k is a constant (e.g., Boltzmann's constant, T is absolute temperature of the resistor, B is the bandwidth, and R is the resistance. As such, increasing the resistance may increase the noisiness of the detected signal. A tradeoff may thus exist in obtaining the optimum resistance and number of turns for use in the leakage detection circuit 560. As such, the number of turns, N, and resistance may vary in different implementations.

The sensed voltage, Vₛₑₙₛₑ, is provided to leakage detection circuit 560. Leakage detection circuitry 560 may analyze the sensed voltage, Vₛₑₙₛₑ, to determine if Vₛₑₙₛₑ exceeds a predefined threshold, T. In an embodiment, leakage detection circuit 550 may provide to a leakage control unit 562 an indication of whether Vₛₑₙₛₑ exceeds T as well as telemetry data regarding Vₛₑₙₛₑ (e.g., the value of Vₛₑₙₛₑ).

In an embodiment, if the sensed voltage, Vₛₑₙₛₑ, exceeds T, leakage control unit 562 may send control information 578 to the stimulator unit 520 to direct the main implant unit 534 to take some corrective action. This corrective action may include disconnecting or disabling certain implant electronics (e.g., stimulator unit 520, power supply circuitry 539, battery, electrodes 548, or a portion of same). Or, for example, the corrective action may involve the leakage control unit 562 directing the stimulator unit 520 to apply compensation to balance the currents on the wires 550. In one such example, stimulator unit 520 may send an inverse compensation current through one or more electrodes 548.

In yet another example, the corrective action may include the leakage control unit 562 directing the stimulator unit 520 to adjust a duty cycle used by electrode current generators included, for example, in main implant unit 534 for application of stimulation by electrodes 548. Or, in yet another embodiment, the corrective action may include the leakage control unit 562 sending control information 578 to the stimulator unit 520 directing the stimulator unit 520 to adjust (e.g., shorten) the duration of stimulation (e.g. the pulse duration, or number of pulses in a stimulation burst) applied by one or more of electrode contacts 548.

Or, for example, the corrective action may include the main implant unit 534 transmitting a notification to a sound processor (e.g., sound processor 126 of FIG. 1) that current leakage has been detected. The sound processor may receive this notification and then provide an alarm (e.g., an audible or visual alarm) informing the recipient that an unsafe level of current leakage has been detected. This notification may include telemetry information 579, such as the magnitude of the detected current leakage (e.g., small, medium, large leak detected). An embodiment in which leakage detection circuitry 560 provides telemetry information is discussed below.

In another example, leakage control unit 562 may trigger a battery-disconnect action if the signal(s) indicates that sensed voltage exceeds the predefined threshold. For example, leakage control unit 562 may implement a battery-disconnect action that disconnects an on-board power supply to prevent or reduce damage to the implanted circuit and/or surrounding tissue of the implantee, such as described in PCT Application No.: PCT/AU2009/001344 entitled “Power Control for a Medical Implant,” which claims priority to Australian Provisional application No. 2008905254, which are hereby incorporated by reference herein.

In yet another example, the leakage control unit 562 may transmit a message to the implantee providing telemetry data and/or alarms 579. This message may be transferred via coil 536 to the external coil 130 (FIG. 1) and provided to the implantee by sound processor 126 (FIG. 1). In an embodiment, leakage control unit 562 may provide the telemetry data and/or alarm messages to secondary coil interface 532. Secondary coil interface 532 may then transfer the telemetry information and/or alarm messages to the external component (142) using a technique such as load modulation, which involves modulating the load placed on secondary coil 536. Or, secondary coil interface 532 may use, for example, a time division multiplexing type of scheme in which a time periods are specified for transferring/and receiving data. In such an implementation, secondary coil interface 532 may transfer the telemetry information and/or message to the external component 142 during the time period(s) dedicated for transferring information from the internal to the external component. Or, in another embodiment, main implant unit 534 may comprise a separate wireless transceiver (e.g., an RF transceiver) that leakage control unit 562 may use for transferring telemetry data or other information to an external unit regarding current leakage detection.

Or, in an embodiment, cochlear implant 100 may use a system for generating and transmitting messages to an implantee such as described in PCT Patent Application No. PCT/AU96/00403, entitled “Apparatus and Method of Controlling Speech Processors and for Providing Private Data Input via the Same.” Additionally, this system may be combined with the system described in PCT Application No. PCT/AU2009/000483, entitled “Sound Processor for a Medical Implant.” Each of these references is hereby incorporated by reference herein.

As noted above, main implant unit 534 also comprises a back-end leakage detection system comprising coil 525, windings 535, sense resistor 536, and leakage detection circuitry 567. As shown, wires 555 pass through core 525 and connect stimulator unit 520 to electrode contacts 548, 549, and 580. This back-end leakage detection system may operate similarly to the above-discussed front-end leakage detection system to detect current leakage by identifying any current imbalance on wires 555.

Stimulator unit 520, leakage control unit 562 and leakage detection circuitry 560 and 567 may be embodied in, for example, a combination of hardware and software. For example, stimulator unit 520 and leakage control unit 562 may comprise one or more ASICs, switches, amplifiers, etc. as appropriate. Further, circuitry 514, 560, 562, and 567 may be embodied in analog and/or digital hardware.

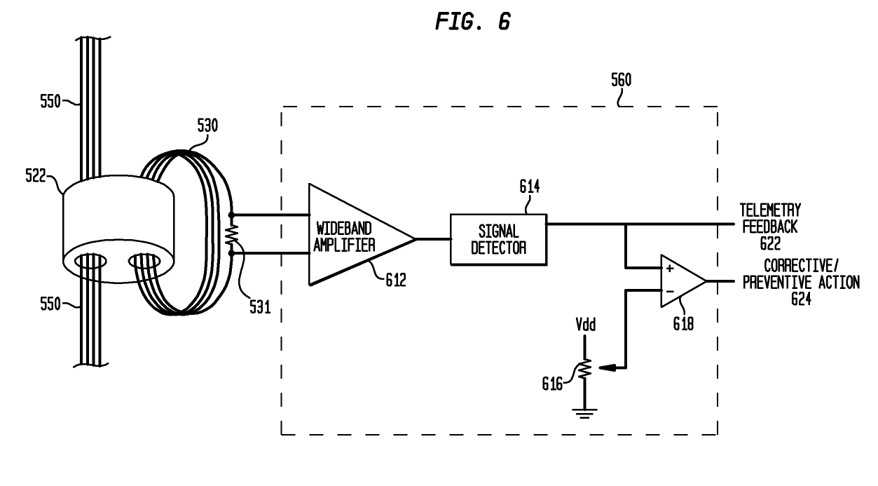

FIG. 6 provides a more detailed illustration of an exemplary leakage detection circuitry, in accordance with an embodiment of the invention. As illustrated, leakage detection circuitry 560 may comprise a wideband amplifier 612, a signal detector 614, a variable resistor 616, and a comparator 618. Further, as shown, leakage detection circuitry 560 may output a telemetry/feedback signal 622 and a corrective/preventive action signal 624. Although FIG. 6 is discussed with reference to the front-end leakage detection system of FIG. 5, it should be understood that similar circuitry may also be employed in back-end leakage detection system.

As illustrated, a wideband amplifier 612 amplifies the voltage, Vₛₑₙₛₑ, across resistor, Rₛₑₙₛₑ, 531 and provides the amplified voltage to a signal detector 614. Wideband amplifier 612 may be a differential amplifier that amplifies the difference in potential across the resistor 531. In an embodiment, wideband amplifier 612 may have a gain-bandwidth product of 1 to 10 MHz and be implemented in, for example, dedicated ASIC technology or commercially available op-amps such as the TSV-911 (from ST Microelectronics) and LM 7321 (from National Semiconductor).

As illustrated, wideband amplifier 612 outputs its signal to a signal detector 614. Signal detector 614 may be any type of device capable of providing a more DC version (i.e., less time varying) of the output of wideband amplifier 614. Signal detector 614 may be used to help reduce the likelihood of false positives resulting from noise by converting the time-varying output of the wideband amplifier 612 to a DC or more DC-like signal. In an embodiment, signal detector 614 may be a series RF diode peak detector. Such an RF diode peak detector may be constructed using, for example, an HSMS-286x Schottky diode. Peak detectors are well known by those of skill in the art and, as such, are not described further herein. It should be noted, that signal detector 614 need not be a peak detector and in other embodiments may be quasi-peak detector, an rms detector, a circuit that outputs a weighted average of its input, etc. Additionally, in embodiments, the signal detector may comprise a filter (e.g., on its input) that may low pass filter the signal from the wideband amplifier. This filter may help reduce the impacts of noise on the signal and have, for example, a cut-off frequency of 50 Hz.

Signal detector 614, as shown, is connected to a comparator 618 that compares the signal from detector 614 against a threshold voltage and outputs a corrective/preventive action signal 624. This threshold voltage may be adjustable using a variable resistor (also referred to as a potentiometer) where the maximum voltage is V_{dd} and the minimum voltage is 0 (ground).

If the output of detector 614 exceeds the threshold, the comparator 618 outputs a corrective/preventive action signal 624 with a value of 1, which indicates that excessive current leakage has been detected. Otherwise, comparator 618 outputs a zero. Comparator 618 may be constructed, for example, using low power circuits (e.g., the LPV 7215 low power comparator from National Semiconductor) or digital systems components, such as an analog to digital (A/D) converter interfaced to a microcontroller. Comparators are well known to those of skill in the art and as such are not described further herein.

As illustrated, leakage detection circuitry 560 provides two outputs: corrective/preventive action signal 624 and a telemetry/feedback signal 622. Telemetry/feedback signal 622, as illustrated, is the output of signal detector 614. Each of these signals may be provided to leakage control unit 562, which may take some corrective action based on these signals, such as discussed above with reference to FIG. 5.

Since wideband amplifiers consume power (e.g., 1 mA/3V), leakage detection circuitry 560 may monitor the sensed voltage, Vₛₑₙₛₑ, under a low duty cycle. For example, leakage detection circuitry 560 may sample the sensed voltage, Vₛₑₙₛₑ, at discrete times, thus enabling the leakage detection circuitry 560 to only power on the wideband amplifier during the time frame when Vₛₑₙₛₑ is to be sampled.

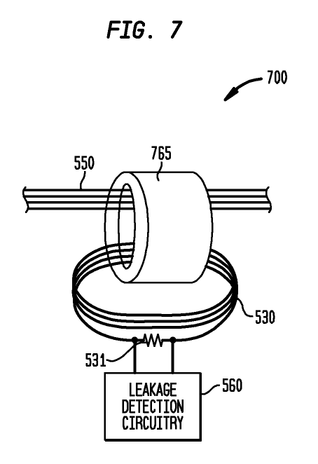

Although the embodiment of FIG. 5 was illustrated with a ferrite core 530 having a “pig nose” shape, it should be noted that in other embodiments ferrite core 530 may have other shapes, such as cylindrical, toroidal, shell, etc. FIG. 7 illustrates a leakage detection system 700 comprising a ferrite core 720 having a cylindrical shape, in accordance with an embodiment of the invention. In system 700 all other components may be identical to those discussed above with reference to FIG. 5. For example, system 700 may comprise wires 550 passing through ferrite core 720. A secondary winding 530 may similarly pass through core 720. This secondary winding may be connected to a sense resistor 531, and the voltage across sense resistor 531 provided to leakage detection circuit 560. Although the above-discussed embodiments were discussed with reference to a ferrite core, in other embodiments other types of core materials may be used, such as, for example, laminated steel, silicon steel, powdered iron, etc.

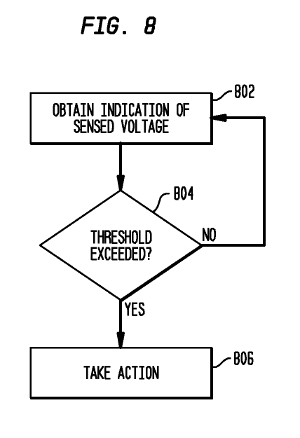

FIG. 8 provides a high level flow chart of i.e. the state machine of the leakage control unit illustrating operations that may be performed in detecting current leakage, in accordance with an embodiment. This method will be discussed with reference to the above-discussed FIGS. 5 and 6. As illustrated, at block 802 an indication of the sensed voltage is received. This indication may be received by leakage control unit 562 from leakage detection circuitry 560 and may comprise for example, corrective/preventive action signal 624 and/or a telemetry/feedback signal 622. At block 804, leakage control unit 562 determines if the threshold, T, has been exceeded. As noted above with reference to FIGS. 5 and 6, leakage detection circuitry 560 may output a corrective/preventive action signal 624 that has a value of “1” if the threshold, T, has been exceeded. In other embodiments, leakage detection circuitry 560 may not include a comparator and instead provide the output of the signal detector 614 directly to the leakage control unit 562. In such an embodiment, leakage control unit 562 may analyze the received signal to determine if the received signal exceeds a predefined threshold.

If the sensed voltage exceeds the threshold, leakage control unit 562 may take some corrective and/or preventive action at block 806. As noted above, this action may include disconnecting certain electronics, directing the stimulator unit 120 to apply compensation/balancing circuits, sending an alarm to sound processor 126, etc. If the sensed voltage does not exceed the threshold, the state machine of the leakage control unit 562 may return to block 802 and continue monitoring the sensed voltage.

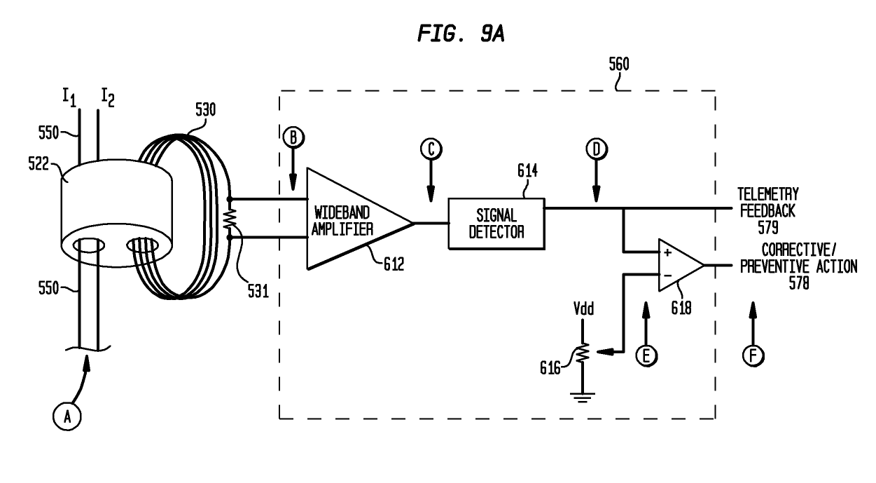

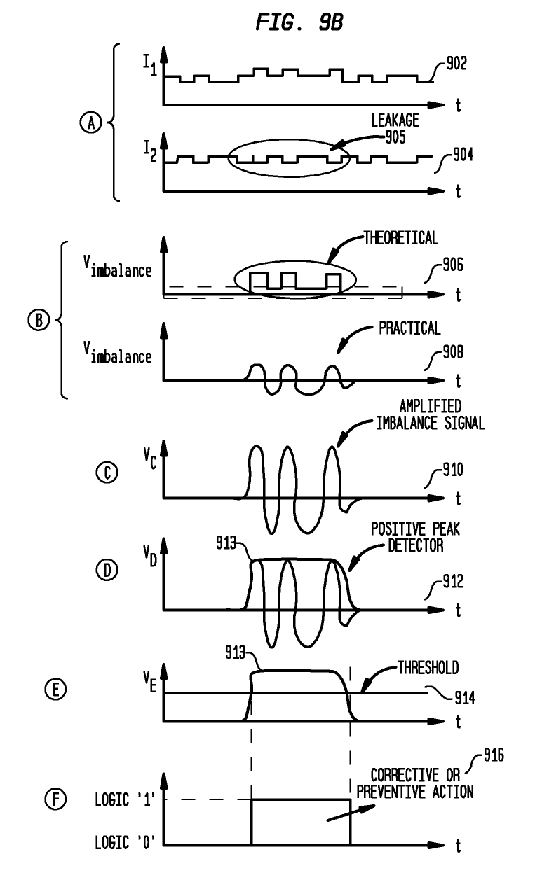

FIGS. 9A and 9B together are provided to provide a simplified explanation of the operation of the leakage detection circuitry 560 of FIG. 6, in accordance with an embodiment of the invention. FIG. 9A illustrates leakage detection circuitry 560 along with arrows pointing to various points, A-F, along leakage detection circuit 560. FIG. 9B illustrates the signals at these points during a situation in which a current imbalance exists due to current leakage. For ease of explanation, wires 550 in FIG. 9A only include two wires, one carrying current, I₁, entering the main implant unit 134 and one carrying current, I₂, exiting main implant unit 134.

FIG. 9B illustrates at point A, the waveforms 902 and 904 for the currents I₁ and I₂ along wires 550. As seen at point 905, these currents are not balanced due to some leakage of current somewhere in the system. At point B, the imbalance is seen when I₁ and I₂ are summed and the resulting current passes through resistor 531 resulting in a sensed voltage. The resulting voltage is illustrated by V_{imbalance} curve 906, which shows the theoretical resulting sensed voltage, and V_{imbalance} curve 908, which illustrates a more practical version of the sensed voltage. As shown in curves 906 and 908, the imbalance in currents I₁ and I₂ results in a non-zero sensed voltage.

At point C, the V_{imbalance} has been amplified by wideband amplifier 612 as shown by curve 910. The output of signal detector (in this case a peak detector) at point D is illustrated by curve 912. As shown in curve 912, the amplified V_{imbalance} has been shaped by its positive envelope to provide a single pulse 913. This pulse 913 is provided to comparator 618, which compares the pulse 913 with the threshold signal 914 at point E. As noted above, this threshold 914 may be adjusted using variable resistor 616. The output of comparator 618 (i.e., the corrective/preventive action signal 624) at point F is illustrated by curve 916, which contains a pulse with a logical value of “1” where the signal 912 exceeds the threshold 914 and curve 916 has a logical value of “0” where signal 912 falls below the threshold. As noted above, the logical value of “1” for the corrective/preventive action signal 624 indicates that an unacceptable current leakage has been detected. This signal 624 may be used to take corrective and/or preventive action as discussed above.

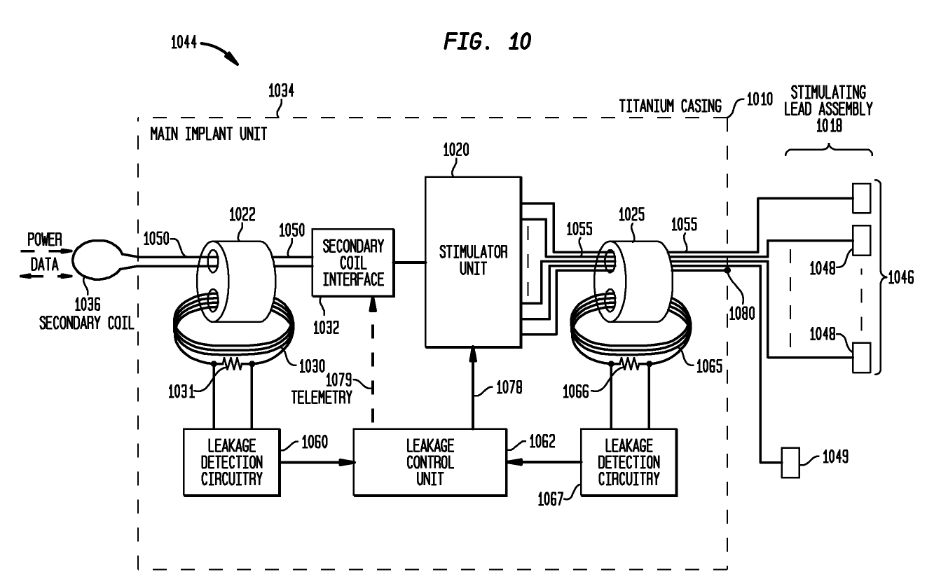

FIG. 10 is a schematic overview of an internal component of a cochlear implant system comprising a current leakage detection system on wires passing between a main implant unit and a secondary coil, in accordance with an embodiment of the present invention. FIG. 10 is similar to the embodiment of FIG. 5 with the exception that the embodiment of FIG. 10 does not include a separate auxiliary implant unit (e.g., auxiliary implant unit 512 of FIG. 5). For example, as shown, implanted component 1044 comprises a main implant unit 1034, a secondary coil 1036, and a stimulating lead assembly 1018. These components may be similar to the similarly named components discussed above with reference to FIG. 1.

As shown, main implant unit 1034 is connected to secondary coil 1036 and stimulating lead assembly 1018. Stimulating lead assembly 1018 comprises an array 1046 of electrode contacts 1048. Also, as shown, main implant unit is connected to a first extra-cochlea electrode 1049 and a second extra-cochlea electrode 1080. The second extra-cochlea electrode 1080 may be mounted on the casing 1010 of main implant unit 1034. Main implant unit, as illustrated, comprises a secondary coil interface 1032 and a stimulator unit 1020. Each of these components may be, for example, identical or similar to the components discussed above with reference to FIGS. 1 and 5.

As shown, main implant unit 1034 comprises front-end leakage detection system comprising a ferrite core 1022, a secondary winding 1030, a resistor 1031, leakage detection circuitry 1060. Main implant unit 1034 also comprises a back-end leakage detection system comprising a ferrite core 1025, a secondary winding 1065, a resistor 1066, leakage detection circuitry 1067. As illustrated, leakage detection circuitry 1060 and 1067 are connected to a leakage control unit. Each of these components of main implant unit 1034 may be, for example, identical or similar to the similarly named components discussed above with reference to FIG. 5.

In the embodiment of FIG. 10, wires 1050 connect secondary coil 1036 to secondary coil interface for transferring and/or reception of power and data. Secondary coil interface 1032 may separate the received power and data. The data may be used to specify the stimulation to be applied as discussed above with reference to FIG. 1. The received power may be used main implant unit 1034.

The leakage detection systems of FIG. 10 may function identically to those discussed above with reference to FIGS. 5-6. For example, as discussed above, if either the front-end or back-end leakage detection system detect a current imbalance, leakage control unit 1062 may take a corrective action, such as discussed above.

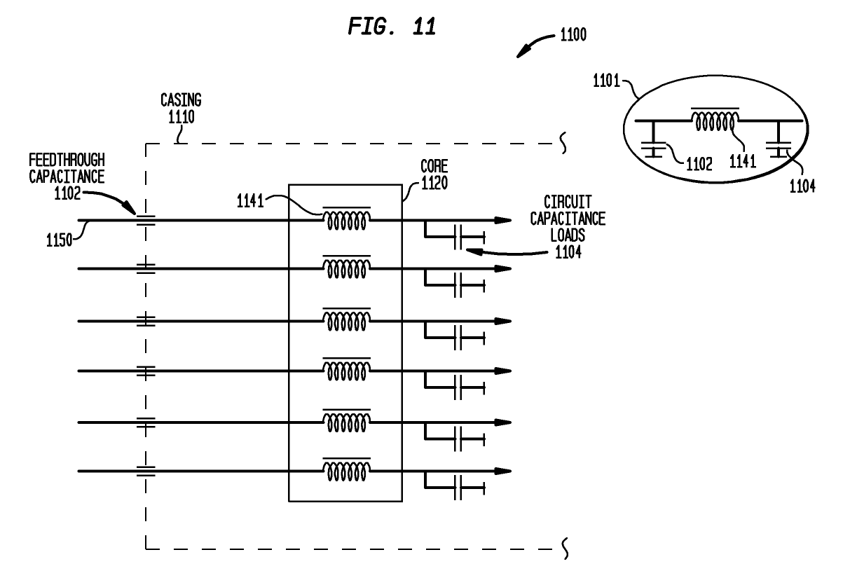

In addition to current leakage detection, the above discussed embodiments may also contribute to the low pass filtering of the signals passing through the core. FIG. 11 provides a simplified illustration of a system capable of providing low pass filtering, in accordance with an embodiment of the invention. The system 1100 of FIG. 11 may be identical to the above discussed system of FIG. 5. As shown, wires 1150 enter casing 1110 and pass through ferrite core 1120. Casing 1110 may be a hermetically sealed casing comprising a feedthrough through which wires 1150 enter/exit casing 1110. This feedthrough may have a capacitance illustrated by feedthrough capacitance 1102. Additionally, the stimulation circuitry and other components of the main implant unit may impart a capacitance on the wires exiting core 1120. This capacitance on each wire is illustrated by circuit capacitance loads 1104. Additionally, an inductance may be imparted on the wires 1150 by the wires passing through core 1120.

The combination of the feedthrough capacitance 1102, inductance 1141, and circuit capacitance 1104 effectively results in a capacitor-input filter 1101 illustrated in the top right corner of FIG. 11. This effective circuit has the effectively impact of applying a low pass filter on the signals of wires 1150. In an embodiment, both the inductance and capacitance may be small (e.g., a capacitance in the range of 1-20 picofarads). In other embodiments, rather than relying on the natural capacitance of the feedthrough and circuitry, precise capacitors could be added to the wires to more precisely control the low pass filtering of the signals on wires 1150. It should be understood that FIG. 11 is a simplified diagram to illustrate the potential low pass filtering imparted by the above discussed embodiment of FIG. 5, and that other parasitic capacitance may exist. For example, a capacitance may also exist between the ferrite core and the implant casing 1110.

The low pass filtering of the system described with reference to FIG. 11 may help offer additional protection to the implant and recipient from damage caused by excess fields (e.g., excessive MRI fields) excess electro magnetic interference (EMI) and/or electro static discharges (ESD) transients. For example, an MRI scanner operating at 1.5 emits a strong pulsed RF (˜64 MHz) signal. The low pass filtering, as shown in FIG. 11, may help attenuate or block these higher frequency signals and thus prevent these signals from entering the implant.

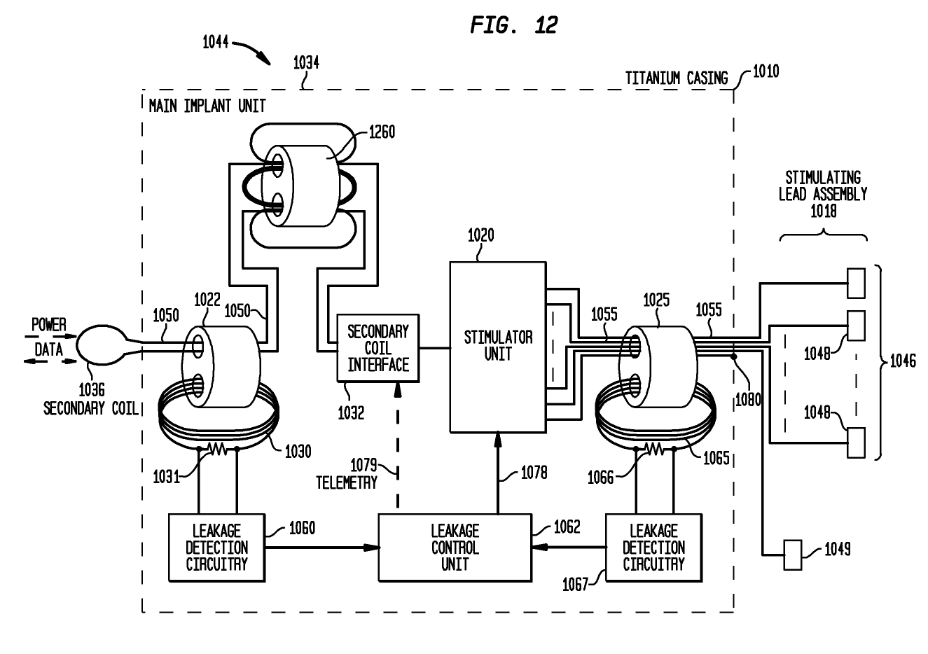

FIG. 12 illustrates an exemplary embodiment of a main implant unit comprising a leakage detection system in combination with a balun, in accordance with an embodiment. FIG. 12 is similar to the embodiment of FIG. 10 with the exception that the embodiment of FIG. 12 comprises a balun 1260. For ease of explanation, the same reference numerals are used in FIG. 10 for identifying corresponding components to those discussed above with reference to FIG. 10. For example, as shown implanted component 1044 comprises a secondary coil 1036, a main implant unit 1034, and a stimulating lead assembly 1018. A secondary coil interface 1032 of main implant unit 1034 is connected to secondary coil 1036. Stimulating lead assembly 1018 comprises an array 1046 of electrode contacts 1048. Each of these components may be, for example, identical or similar to the components discussed above with reference to FIG. 10. As shown, main implant unit 1034 comprises a front-end and back-end leakage detection system each sharing a common leakage control unit 1062. Each of these components of main implant unit 1034 may be, for example, identical or similar to the components discussed above with reference to FIG. 10.

In the embodiment of FIG. 12, main implant unit 1034 also comprises a balun 1060 through which wires 1050 pass. It should be noted that FIG. 12 is a simplified schematic diagram and the illustration of balun 1060 is merely provided to indicate that the embodiment may comprise a balun 1060, and not to endorse a particular type, shape, or configuration of balun 1060.

In the embodiment of FIG. 12, balun 1060 may be an RF balun designed to help balance the signal at RF frequencies. For example, in the illustrated embodiment, the wires 1050 are connected to coil 1036 for transmission and/or reception of power and data, as discussed above with reference to FIG. 10. This transmission/reception of power/data via coil 1036 may occur at RF frequencies (commonly defined as the frequencies between 9 kHz to 300 GHz), such as for example at a frequency of between 2.5 MHz to 5 MHz, or a frequency of approximately 50 MHz. In embodiments, an RF balun may be useful to help balance the received signals at RF frequencies. Such an RF balun, however, may not impact the imbalance(s) resulting from current leakage, which may be at a frequency much lower than the RF frequencies used for transmission/reception. In other embodiments, an RF balun may not be used in the system of FIG. 12. Or, in other embodiments, an isolation transformer may be included in main implant unit 1034 to help improve safety. RF baluns and isolation transformers are well known to those of skill in the art, and as such, are not described further herein.

In another embodiment, the leakage detection systems may be placed elsewhere in the system. For example, in other embodiments, only a front-end leakage detection system may be used, or only a back-end leakage detection system may be used. Or, for example, the front-end and back-end leakage detection systems may be combined. For example, in an embodiment, a single core (and accordingly common leakage detection circuitry and a common leakage control unit) may be used and all wires entering/exiting the main implant unit may pass through this single core (e.g., the wires connecting the main implant unit to the electrode contacts, to the secondary coil, and/or to the auxiliary implant unit).

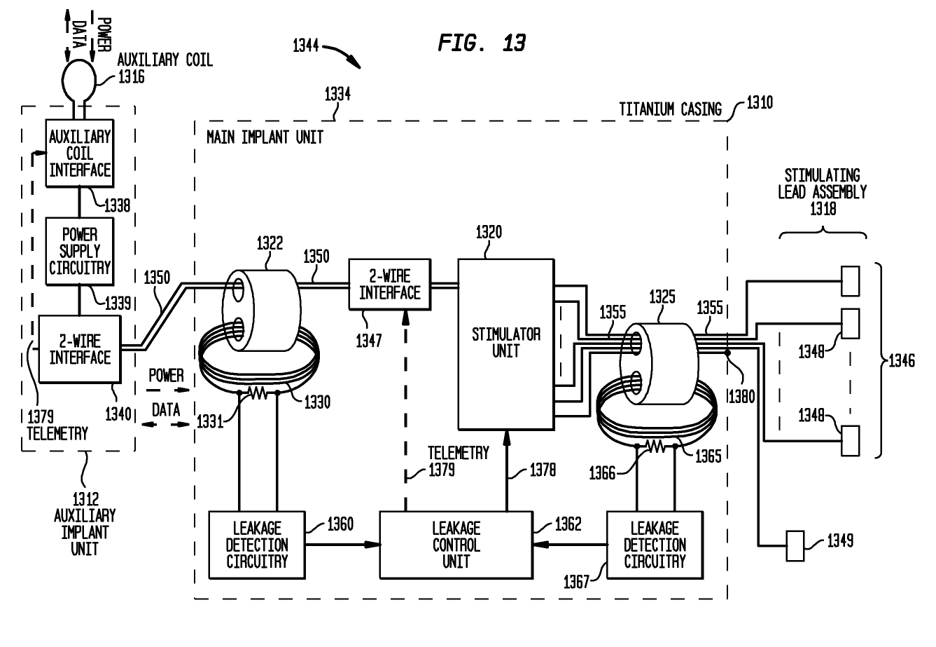

The above-discussed leakage detection system may be used in other embodiments. For example, FIG. 13 illustrates is a schematic overview of an internal component of a cochlear implant system comprising a current leakage detection system on wires passing between the main implant unit and an auxiliary implant unit, in accordance with an embodiment of the present invention.

FIG. 13 is similar to the embodiment of FIG. 5 with the exception that the embodiment of FIG. 13 does not include a separate secondary coil. For example, as shown implanted component 1344 comprises auxiliary implant unit 1332, a main implant unit 1334, and a stimulating lead assembly 1318. Stimulating lead assembly 1318 comprises an array 1346 of electrode contacts 1348. Additionally, as shown, a first extra-cochlea electrode 1349 and a second extra-cochlea electrode 1380 is connected to a stimulator unit 1320 of main implant unit 1334. Further, as illustrated, main implant unit 1334 comprises a 2-wire interface 1347, a stimulator unit 1320, and a front-end and back-end leakage detection system. The front-end leakage detection system comprises a ferrite core 1322, a secondary winding 1330, a resistor 1331, leakage detection circuitry 1360, and a leakage control unit 1362. The back-end leakage detection system comprises a ferrite core 1325, a secondary winding 1365, a resistor 1366, leakage detection circuitry 1367, and shares leakage control unit 1362 with the front-end leakage detection system. Each of these components may be, for example, identical or similar to the components discussed above with reference to FIGS. 1 and 5. As illustrated, leakage control unit 1362 may output a telemetry signal 1379 and/or a control signal 1378 similar to the above-discussed embodiments.

Auxiliary implant module 1312 may comprise a hermetically sealed housing that houses a power source, such as rechargeable battery, a microphone, and/or other electronics. In the illustrated embodiment, auxiliary implant unit 1312 is an auxiliary implant module 512, such as discussed above with reference to FIG. 5. In the illustrated embodiment, leakage control unit 1362 may transfer telemetry data (or other information) to an external unit by providing the telemetry data 1379 to the 2-wire interface 1347 which provides the telemetry data 1379 to the auxiliary implant unit 1312 for transferring the data to an external unit. Such an external unit may be, for example, a sound processor, a remote unit etc. For example, as discussed above, the external unit may use the received telemetry data (or, e.g., an alarm notification) from the leakage control unit 1362 to notify (e.g., generate a visual and/or audible alarm) the recipient of current leakage in the event current leakage is detected.

Or, in another embodiment, the internal components 1344 of FIG. 13 may be for a totally implantable cochlear implant. In such an embodiment, auxiliary implant module 1312 may comprise electronics and circuitry that is more likely to need replacement during the lifetime of the recipient. This would enable a surgeon to replace just the auxiliary implant module 1312 without the need for surgically removing and/or replacing the main implant unit 1334 or stimulating lead assembly 1318.

It should be understood that the embodiment of FIG. 13 is provided to illustrate how a leakage detection system may be used to check for current leakage causing imbalances on wires connecting to other types of implanted components. Further, the specific functions of auxiliary implant module 1332 may be different in different implementations.

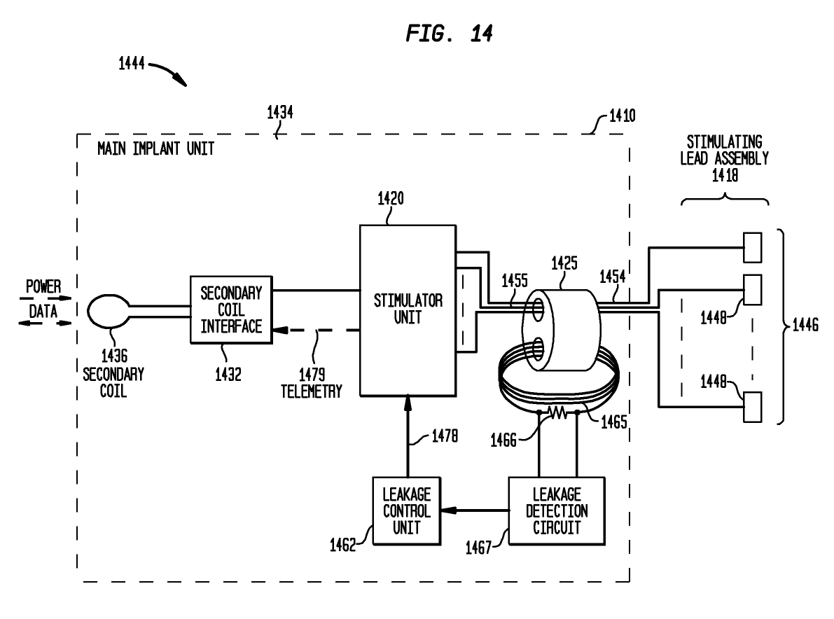

In yet another embodiment, a current leakage detection system may be implemented in a cochlear implant system in which the secondary coil is located within the housing of the main implant unit. FIG. 14 illustrates is a schematic overview of an internal component of a cochlear implant system in which the secondary coil is located within the housing of the main implant unit, in accordance with an embodiment of the invention. The embodiment of the FIG. 14 may be similar to the embodiment of FIG. 10, with the exception that the secondary coil 1436 is located within the housing 1410 of main implant unit 1434. Further, because the secondary coil 1436 is located within housing 1410, no wires enter or exit housing 1410 in connecting secondary coil interface 1332 to secondary coil 1336. The housing 1410 may be a heremetically sealed biocompatible housing. For example, in an embodiment, housing 1410 may be manufactured from an organic polymer thermoplastic such as polyether ether ketone (PEEK). Or, for example, housing 1410 may be a ceramic housing.

In the illustrated embodiment of FIG. 14, a front-end leakage detection system is not employed. Rather, only back-end leakage detection system is used. As shown, the back-end leakage detection system checks for current imbalances on wires connecting stimulator unit 1420 of the main implant unit 1434 to electrode contacts 1448 in an array 1446 of electrode contacts of stimulating lead assembly 1418. In the illustrated embodiment, extra-cochlea electrodes are not used. For example, the embodiment of FIG. 14 may employ bipolar, tri-polar, and/or phased-array stimulation.

In the illustrated embodiment, the back-end leakage detection system comprises a core 1425, a secondary winding 1465, a resistor 1466, leakage detection circuitry 1467, and a leakage control unit 1462. Each of these components may function in a similar manner to the like-named components discussed above with reference to FIGS. 5, 6, and 10. For example, core 1425 may be a ferrite core, and leakage control unit 1462 may output telemetry data 1379 and a control signal 1478. Leakage control unit 1462 may perform a corrective action, such as discussed in the above embodiments, if current leakage is detected. In the presently described embodiment, leakage control unit 1462 may provide information 1478 to stimulator unit 1420. This information may comprise telemetry data, instructions for performing a corrective action, a message (e.g., an alarm) to be transmitted to an external device, etc. If information (e.g., telemetry data 1479) is to be transmitted to an external unit, stimulator unit 1420 may provide the telemetry data 1479 to secondary coil interface 1432 for transference to the external unit. Secondary coil interface 1432 may use any technique, such as those discussed above (e.g., load modulation) for transferring the data to the external device.

The above described embodiments of a leakage detection system may offer a number of advantages. For example, the above discussed leakage detection system may be able to detect small AC/RF current leakages in the tissue due to a malfunction (e.g., a failure) of the implant. These malfunctions could potentially result in adverse effects such as a pain sensation, excessive nerve stimulation, or tissue damage. Detection of these leakages and taking appropriate corrective action may help improve the safety of the medical device.

Additionally, the leakage detection system may also, in certain embodiments, be able to detect high AC/RF current leakages caused by MRI scanners, high EMI or ESD. By detecting these leakages, appropriate corrective action may be taken, such as activating an implant protection circuit deactivating the implant, or sending a notification message.

Further, in embodiments, the leakage detection system is electrically isolated from the electrical conducts (e.g., via galvanic separation). Thus, it may be easier to connect (e.g., interface) the leakage detection system to existing circuitry, such as ASICs already included in existing implant systems.

All documents, patents, journal articles and other materials cited in the present application are hereby incorporated by reference.

Embodiments of the present invention have been described with reference to several aspects of the present invention. It would be appreciated that embodiments described in the context of one aspect may be used in other aspects without departing from the scope of the present invention.

Although the present invention has been fully described in conjunction with several embodiments thereof with reference to the accompanying drawings, it is to be understood that various changes and modifications may be apparent to those skilled in the art. Such changes and modifications are to be understood as included within the scope of the present invention as defined by the appended claims, unless they depart there from.

What is claimed is:

1. An implantable medical device comprising:
at least one electronic circuit;
a current imbalance detector comprising:
a core surrounding at least a portion of one or more electrical conductors connected to the at least one electronic circuit;
a winding on the core; and
a detection circuit connected to the winding and configured to provide a signal indicative of whether there is an imbalance in current conducted by the one more electrical conductors; and
a hermetically sealed housing that houses said at least one electronic circuit and said current imbalance detector.

2. The implantable medical device of claim 1, wherein the detection circuit comprises:
a resistive element connected to the winding, wherein a voltage across the resistive element provides the signal indicative of whether there is a current imbalance.

3. The implantable medical device of claim 2, wherein the voltage across the resistive element is proportional to a common mode current through the one or more electrical conductors and a number of turns of the winding on the core.

4. The implantable medical device of claim 2, wherein the resistive element comprises a resistor connected to the winding, and wherein the resistor is connected to an input of an amplifier.

5. The implantable medical device of claim 2, wherein the resistive element comprises an intrinsic resistance of an input of an amplifier.

6. The implantable medical device of claim 1, wherein the detection circuit comprises a signal detector configured to provide an output based on a sensed signal from the winding.

7. The implantable medical device of claim 1, wherein the detection circuit comprises an amplifier configured to amplify the current imbalance.

8. The implantable medical device of 1, further comprising a comparator configured to provide an output indicative of whether the output of the current imbalance detector exceeds a threshold.

9. The implantable medical device of claim 1, further comprising:
a leakage control unit configured to receive the signal indicative of whether there is a current imbalance and perform an action based on the received signal.

10. The implantable medical device of claim 9, wherein the leakage control unit is configured to take a corrective action if the signal indicates that there is a current imbalance;
wherein the corrective action includes at least one of disconnecting one or more electrical components of the medical device, disconnecting a power supply; directing a compensation current to be applied; adjusting a duty cycle; and/or transmitting an alarm message.

11. The implantable medical device of claim 1, further comprising:
an RF balun.

12. The implantable medical device of claim 1, wherein the implantable medical device is an implantable component of a cochlear implant system.

13. The implantable medical device of claim 1, wherein the implantable medical device is an active implantable medical device.

14. The implantable medical device of claim 13, wherein the active implantable medical device contains an implantable component configured to give electrical stimulation and/or electro-mechanical stimulation.

15. The implantable medical device of claim 1, wherein the implantable medical device is an implantable component for a system selected from the set of an functional electrical stimulation system, an electro-mechanical stimulation system, an auditory brainstem system, a spinal cord stimulator system, a heart stimulation system, a drug dispensing system, and a bone growth stimulation system.

16. The implantable medical device of claim 1, wherein the core is a ferrite core.

17. The implantable medical device of claim 1, wherein the signal indicative of whether there is a current imbalance is a signal that has a specified logical value if a current imbalance is detected, the implantable medical device further comprising:
a transmission device configured to transfer the signal to an external device.

18. The implantable medical device of claim 1, wherein the signal indicative of whether there is a current imbalance is a signal representative of a value of any current imbalance on the one or more electrical conductors, the implantable medical device further comprising:
a transmission device configured to transfer the signal to an external device.

19. A method for use in an implantable medical device having at least one electronic circuit, the method comprising:
obtaining a signal representative of a sensed signal from a winding on a core, wherein the core surrounds at least a portion of one or more electronic conductors connected to the at least one electronic circuit of the implantable medical device;
determining if the sensed signal indicates an imbalance in current conducted by the one or more electrical conductors; and
performing a corrective action if a current imbalance is detected exceeding a threshold.

20. The method of claim 19, wherein determining if the sensed signal indicates an imbalance in current comprises:
amplifying the sensed signal; and
comparing said amplified sensed signal with the threshold.

21. The method of claim 19, wherein the corrective action includes at least one of:
disconnecting or disabling one or more electrical components of the medical device;
disconnecting or disabling a power supply;
directing a compensation current to be applied; adjusting a duty cycle; and
transmitting an alarm message.

22. The method of claim 19, wherein the implantable medical device is a component of a cochlear implant system.

23. The method of claim 19, wherein the implantable medical device is an active implantable medical device.

24. The method of claim 19, wherein the implantable medical device contains an implantable component giving electrical stimulation and/or electro-mechanical stimulation.

25. The method of claim 19, wherein the active implantable medical device is at least one of an function electrical stimulation system, an electro-mechanical stimulation system, an auditory brainstem system, a spinal cord stimulator system, a heart stimulation system, a drug dispensing system, and a bone growth stimulation system.

26. A system for use in an implantable medical device having at least one electronic circuit, the system comprising:
means for obtaining a signal representative of a sensed signal from a winding on a core, wherein the core surrounds at least a portion of one or more electronic conductors connected to the at least one electronic circuit of the implantable medical device;
means for determining if the sensed signal indicates an imbalance in current conducted by the one or more electrical conductors; and
means for performing a corrective action if a current imbalance is detected exceeding a threshold.
